(19) United States
(12) Patent Application Publication
(10) Pub. No.: US 2026/0319129 A1
(43) Pub. Date: Oct. 15, 2026

(54) METHOD FOR DETERMINING RESIDUAL OIL SATURATION (SOR) BY MEASURING THE RELATIONSHIP BETWEEN SOR, SW AND PVI

(71) Applicant: PETRÓLEO BRASILEIRO S.A. - PETROBRAS, Rio de Janeiro (BR)

(72) Inventors: Filipe Ramos De Albuquerque, Rio de Janeiro (BR); Joao Paolo Teixeira Da Fonseca, Rio de Janeiro (BR); Gabriel Luiz Perez Vieira, Santos (BR)

(21) Appl. No.: 19/179,752

(22) Filed: Apr. 15, 2025

(30) Foreign Application Priority Data
Apr. 19, 2024 (BR) 1020240076907

(51) Int. Cl. G01V 20/00; G01N 33/24

(52) U.S. Cl. CPC G01V 20/00; G01N 33/241; G01N 33/246

(57) ABSTRACT

The present invention proposes a method for determining the residual hydrocarbon saturation (SOR) at the well scale based on the relationship between water saturation (SW), water injection pore volume (PVI) due to invasion of drilling mud filtrate in wells drilled with water-based drilling mud (WBM) and residual hydrocarbon saturation (SOR). The present invention further describes a method for determining SOR at the well/log scale by modeling the relationship between Sw and PVI expressed by the function Sw(PVI). The present invention is within the oil industry field from exploration, for reserve estimation and declaration of commerciality, passing through to oil production, where knowing the SOR is necessary to design production and, finally, for estimating the volume of oil that can be produced, recovered or exploited from the reservoir.

### FIELD OF THE INVENTION

The present invention proposes a method for determining the residual hydrocarbon saturation (SOR) at the well scale based on the relationship between water saturation (SW), water injection pore volume (PVI) due to invasion of drilling mud filtrate in wells drilled with water-based drilling mud (WBM) and residual hydrocarbon saturation (SOR).

The present invention is within the oil industry field from exploration, for reserve estimation and declaration of commerciality, passing through to oil production, where knowing the SOR is necessary to design production and, finally, for estimating the volume of oil that can be produced, recovered or exploited from the reservoir.

### BACKGROUND OF THE INVENTION

Residual hydrocarbon or oil saturation (SOR) is one of the main characterization parameters of an oil reservoir rock, being relevant for reserve estimations, production and declaration of commerciality of wells. In this sense, reservoir rock is defined as a subsurface accumulation of oil or gas in porous and/or fractured rocks.

In the oil industry, there is a paradigm of not estimating SOR by profiles in open wells (with no metal production casing) in a non-depleted reservoir, that is, the mobile oil present in the rock has not yet been properly washed away by the injection water from surrounding wells (AI-Housani et al., 2012 Using Residual Oil Saturation (Sor) to Reduce Uncertainty in Reservoir Modeling—A Case Study. SPE 161531, presented at the Abu Dhabi International Petroleum Exhibition and Conference (Abu Dhabi UAE, November 11-14). DOI: 10.2118/161531-MS; Murugesu et al., 2017 Integrated Approach in Determining Residual Oil Saturations to Water Sorw and its Impact on Potential Enhanced Oil Recovery FOR Volume Estimation. SPE/IATMI Asia Pacific Oil & Gas Conference and Exhibition, Jakarta, Indonesia, October 2017. DOI: 10.2118/186906-MS).

However, it is not uncommon that in exploratory oil wells and during the initial stages of production, SOR curves by electrical logging (also known as geophysical well log or petrophysical well log) are usually calculated by applying the ‘Archie equation’ for saturation of the zone invaded by water-based drilling mud filtrate (Sxo), using a microresistivity log. This method estimates SOR directly from Sxo, assuming that SOR would be the difference between the total porous volume of the rock and Sxo (SOR=1−Sxo), which is an inaccurate premise.

In fact, this technique performs an estimate of the remaining oil (ROS) in the rock, since there is no guarantee that the invaded zone has been properly washed until reaching SOR conditions, which, in effect, would denote the remaining presence of mobile oil. In other words, this technique tends to overestimate SOR.

Another SOR estimation method known in the literature is the one that uses the Sigma tool, also known as Pulsed Neutrons Log (PNL). Logging is only performed in oil-producing wells where water production is increasing, indicating that the reservoir is probably already washed by the injection of water from adjacent wells, a common process resulting from oil field production. However, well logging under this condition is rare and late in the production curve.

SOR data can also be obtained from rock samples through laboratory tests (‘KREL’—for determining relative permeability curves—and ‘Capillary Pressure’) and ‘Digital Rock’ methods. However, rock samples are point data (in a scale of millimeters to centimeters) when compared to the reservoir and the aforementioned laboratory analyses are scarce when compared to continuous electrical logging (in a scale of decimeters to meters) and have a different scale than the reservoir and the well, facts that make it difficult to directly upscale from this data.

Direct methods using the injection of natural and artificial tracer liquids through the rock formation can contact the portions of interest thereof. However, due to operational difficulties, artificial tracers do not allow the determination of SOR continuously throughout the useful life of the field.

Thus, the methods available so far in the state of the art are costly and complex to operate, in addition to providing unrepresentative results, and mainly, they cannot be used continuously throughout the useful life of the reservoir, as they prevent an accurate assessment of SOR at the well scale.

Among the state of the art documents that have already addressed the problem, the following patent and non-patent references are cited, for example:

The paper by Podberezhny, M., Polushkin, S., Marketz, F., Makarov, A., Sviridov, M., and A., Mosin. “Determination of Invaded Zone Properties from Time-Lapse Induction Logging Measurements Based on Invasion Simulation and Electromagnetic Modeling.” Paper presented at the SPE Russian Petroleum Technology Conference and Exhibition, Moscow, Russia, October 2016. doi: https://doi.org/10.2118/182103-MS discusses the importance of studying the evolution of the invaded zone, also confirming that modeling the evolution of the invaded zone allows the determination of the true properties of the uninvaded formation. However, the paper is not aimed at estimating the residual oil saturation (SOR). Residual oil saturation appears as input in this paper and the authors use a standardized (fixed) value, without discussing the issue of residual oil saturation (SOR) being variable for each rock volume.

The paper by Murugesu, Thanapala S, Masoudi, Rahim, Johare, Dzulfadly B., Jalan, Shlok, and Izral Izarruddin Marzuki. “Integrated Approach in Determining Residual Oil Saturations to Water Sorw and its Impact on Potential Enhanced Oil Recovery EOR Volume Estimation.” Paper presented at the SPE/IATMI Asia Pacific Oil & Gas Conference and Exhibition, Jakarta, Indonesia, October 2017. doi: https://doi.org/10.2118/186906-MS discusses the definition and high importance of SOR, summarizing the main methods applied by the industry to estimate such petrophysical property of oil reservoirs, highlighting the limitations and uncertainties of each one. However, said paper does not present any new method for estimating SOR, only suggesting that SOR, at the well scale, is only calculated by electrical logging in reservoirs already depleted by oil production. In contrast, the method proposed herein allows estimating SOR by electrical logging in an oil reservoir that is not yet depleted, based on the knowledge of water saturation in the zone invaded by the filtrate of water-based drilling fluid (water-based mud, WBM) and invaded filtrate volume (PVI).

In turn, the paper by Kennaird, T. “Residual Oil Saturations Determined by Core Analysis.” Paper presented at the Offshore South East Asia Show, Singapore, February 1988. doi: https://doi.org/10.2118/17686-MS presents laboratory test data of core samples from a variety of lithologies in the Asia-Pacific region, from which one would already know the inverse relationship between SOR and Sw. However, said paper discloses a study of a laboratory analysis of oil saturation in core samples (plugs of rock cores); therefore, not focusing on the study of SOR at the well scale by electrical logging.

International application PCT/US2018/045632 (publication WO2019/032597) was filed by ARAMCO SERVICES COMPANY on Aug. 7, 2018, and describes a method for determining waterflooding residual oil saturation using a linear regression fit method. However, said document has two differences relative to the present invention: 1) the first consists of the fact that the applicant uses capillary pressure and Sw data obtained in a centrifugation test, whereas in the present invention Sw and water injection volume (PVI) data are used, which are applied at the well scale by correlating with the process of injecting water from drilling fluid filtrate invasion; 2) the second, which arises from the former, consists of the fact that in said document curves are correlated to estimate SORw in the laboratory, in the centrifugal capillary pressure test required to design the SORw value, without the need to take the test to the end point, which would require a lot of time, that is, the objective of that method is not to determine SOR at the well scale, but rather in capillary pressure tests in the laboratory. The present invention discloses a method for determining SOR at the well scale using electrical logging and invaded zone modeling.

Russian patent RU2178515 was filed by ‘000 JARRO’ and ‘ANSKNIPINEFT’ and published in 2002, and describes a method of determining residual oil saturation with a different formula than that described in the present invention. However, said document already predicted that residual oil saturation (S*) in the permeability coefficient is affected by the function S*=1/(1+p·kq), where k is the permeability coefficient; p and q are coefficients. Thus, they deduced that S* (or SOR) would be inversely proportional to k. However, it is worth noting that in the petrophysical environment it is well known that the SOR property, in general, has an inverse relationship to Swi. Low permeability rocks tend to have high Swi, and consequently low SOR. Therefore, this conclusion of the inverse relationship between SOR and permeability is not plausible. However, the method of the present invention does not use correlations with rock permeability, but rather correlations of water saturation (Sw), injected pore water volume (PVI) and residual oil saturation (SOR).

Therefore, the state of the art does not disclose an adequate and reliable method for determining SOR at the well scale, which can be easily applied in petrophysical reservoir evaluation and in simulating reservoir flows.

Therefore, there is a need for a method to accurately estimate SOR at the well scale in reservoirs not depleted by production.

### SUMMARY OF THE INVENTION

In order to address the technical problem of the scarcity of reliable methods for determining residual oil (hydrocarbon) saturation (SOR) at the well scale, the present invention presents a method for determining SOR based on the relationship between Sw and PVI. It is important to note that the accurate determination of SOR is of utmost importance for estimating oil reserves and production.

In a first embodiment, which serves as a theoretical basis for the second, the present invention describes a method for determining SOR at the well/log scale by correlating it with water saturation (Sw), which in a well drilled with water-based drilling fluid (WBM) would basically be the invaded zone water saturation (Sxo) for injected filtrate volume (PVI) intervals.

In a second embodiment, the present invention further describes a method for determining residual oil saturation (SOR) at the well/log scale through the model of the relationship between Sw and PVI expressed by the function Sw(PVI).

The present invention provides for the determination of residual oil saturation (SOR) at the well scale/log quickly and relying solely on well log-scale data, the invaded zone water saturation (Sxo), the irreducible water saturation (Swi) and the volume of injected water into the formation weighted by porosity (PVI).

Advantageously, the present invention provides a method for determining SOR based on water saturation logs from the invaded zone in an appropriate manner and at the log scale, wherein the method can be easily applied in Petrophysical Evaluation of Reservoir, Exploration Opportunities and Reservoir Engineering, such as, for instance, in formation assessment processes in petrophysical reservoir modeling.

Due to the practicality of the method proposed herein, the present invention presents the following advantages:
1) it provides yield gains in the petrophysical evaluation routine;
2) it reduces uncertainty in reserve and production estimates;
3) it contributes to increasing reserve estimates and oil production in the reservoir;
4) it reduces the number of logging operations with tools with radioactive sources in oil wells with the specific purpose of determining SOR.

These goals and other advantages of the invention are better described in the following sections.

### BRIEF DESCRIPTION OF THE FIGURES

In order for the invention to be understood more easily, the following figures are presented byway of illustration, but with no intention to limit the invention, which figures accompany this specification and are an integral part thereof.

FIGS. 1a-1d present three-dimensional plots of SOR, Sw and PVI from laboratory water injection tests (relative water-oil permeability), highlighting the correlation between these three variables.

FIGS. 2a-2t present scatter plots of SW and SOR by Sw intervals by PVI.

FIG. 3 shows the flowchart of the process of estimating SOR using regression relationships.

FIGS. 4a-4b illustrate the relationship between SOR predicted by SW regression by SOR by PVI intervals in the relative oil-water permeability test data compared to SOR measured in the experiment (R²=0.66/(a) scatter plot/(b) 2D histogram plot).

FIGS. 5a-5f show the Sw vs PVI curves of krel tests and Sw(PVI) function curves fitted to the respective experimental data.

FIGS. 6a-6b show the parameter ‘a’ estimated from the Sw(PVI) function for each SW and PVI pair from the experimental data along with the ‘a’ adjusted to the experimental data (R²=0.88/(a) scatter plot; (b) 2D histogram plot).

FIG. 7 shows the flowchart of the process of estimating SOR using the Sw(PVI) function.

FIGS. 8a-8b illustrate SOR predicted by the Sw(PVI) function using the estimated ‘a’ parameter for each SW and PVI pair of the assay and the experimental or ‘measured’ SOR (R²=0.6711/(a) scatter plot; (b) 2D histogram plot).

FIG. 9 shows an instance of well-scale application using log data from both the SOR method by Sw regression by PVI intervals and the application of the Sw(PVI) function.

### DETAILED DESCRIPTION OF THE INVENTION

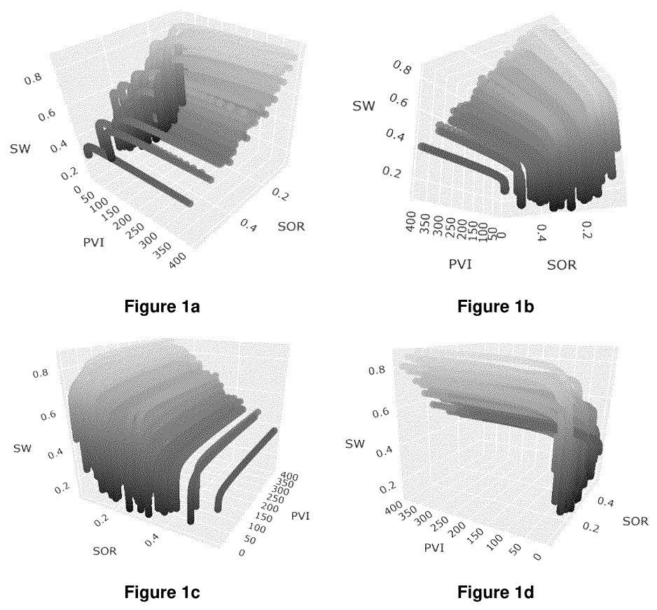

In general terms, the present invention describes a method for determining residual oil saturation (SOR) at the well scale through the relationship between water saturation (Sw) and the volume of water injected into the rock (PVI). This relationship was first observed in laboratory water injection tests (relative water/oil permeability tests under transient conditions) carried out on carbonate samples from the Barra-Velha Formation saturated with oil, as exemplified in FIGS. 1a-1d. This relationship was explored to estimate SOR through Sw vs SOR regressions for PVI intervals, also through a new mathematical function designated as Sw(PVI).

The method proposed by the present invention is intended to determine SOR at a log scale in open-hole wells drilled with water-based mud (WBM), in oil-bearing reservoirs.

The methods based on the intrinsic relationship between Sw and PVI of the present invention are intended to estimate SOR at a well/log scale by considering Sw of the flushed zone (Sxo) as a function of the invasion PVI. The process has low computational cost and is easy to apply, depending only on knowledge of the well scale, the water saturation of the invaded zone (Sxo), the irreducible water saturation (Swi) and the volume of water injected into the formation (PVI).

Application of the method at the well scale depends on the occurrence of oil displacement by water from the drilling fluid (WBM) in a reservoir that is essentially saturated with oil. Both the water saturation in the invaded zone (Sxo) and the volume of injected aqueous filtrate (PVI) are due to the process of aqueous invasion into the rock formation, which takes place due to pressure overbalance. Both quantities can be determined by electrical log inversion, numerical invasion modeling and other methods.

The present invention also provides an analytical method for determining residual oil saturation (SOR) at a well/log scale, based on an original function developed to describe water saturation as a function of the water injected volume (PVI).

The new mathematical function, hereinafter referred to as Sw(PVI), is given by equation 1, as described below:

$$Sw ( PVI ) = \frac{{PVI}_{\sqrt{{PVI}^{a}}}^{a / 2}}{20 * a} + Swi \qquad ( Eq . 1 )$$

wherein:
PVI=injected pore volume;
Sw=water saturation;
Swi=irreducible water saturation;
α=fitting coefficient of the function Sw(PVI)

The coefficient α that fits the Sw(PVI) function is understood as the coefficient of oil displacement by water, so that the greater the coefficient α, the lower the oil displacement by water and, accordingly, the higher the SOR estimated by the function. The above function was achieved from the analysis of more than 100 experimental Sw vs PVI curves, with good fits of the function for each curve, and for which the coefficient was determined α. To obtain the coefficient α (oil displacement by water at a log scale) prior knowledge of the variables Swi, Sw and PVI is required, which quantities, as previously discussed, can be estimated by electrical log inversion, numerical invasion modeling and other methods.

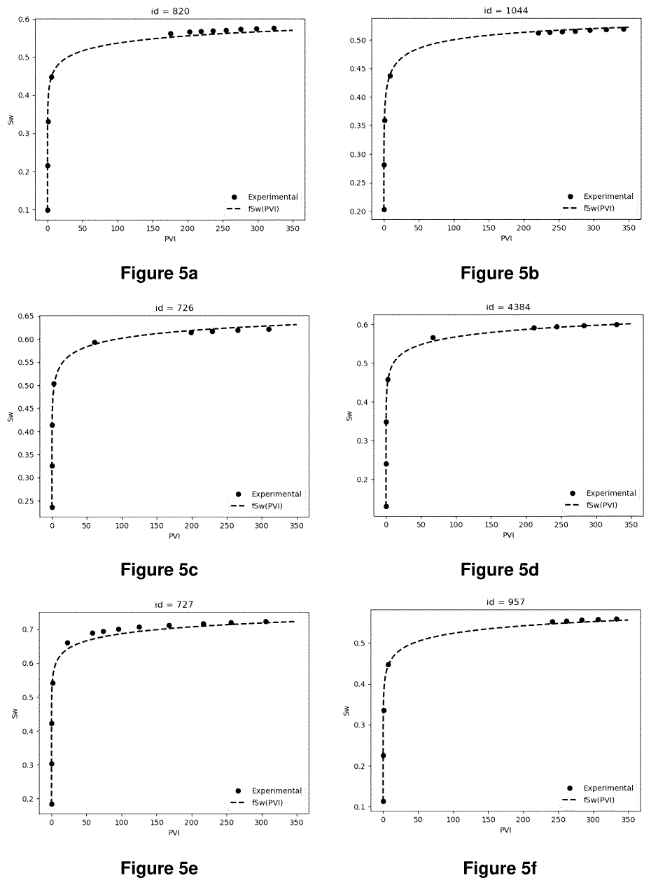

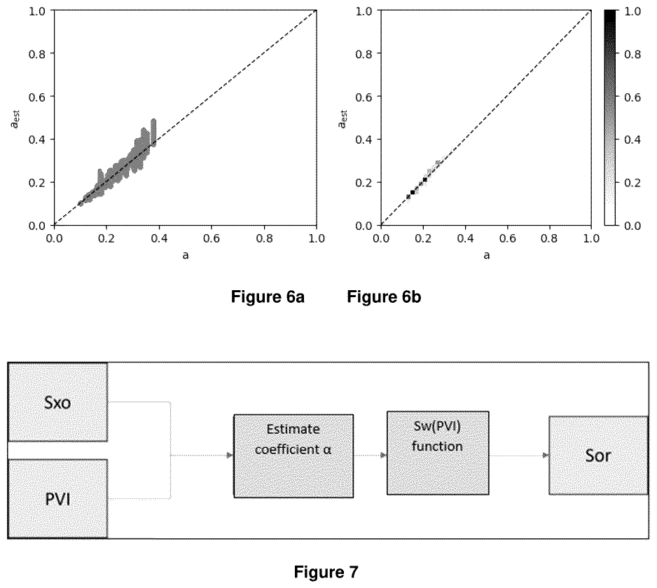

FIGS. 5a-5f show a comparison of coefficient α fitted to the experimental curves and α estimated by equation Sw(PVI) for coefficient α for each pair of Sw and PVI from these same experimental data. The typical interval for values α ranges from about 0.14 and about 0.30. Alternatively, the SOR value can be obtained by any other similar mathematical method, for example, having variations of the Sw(PVI) function, wherein Sw is the dependent variable Sw, and wherein the independent variable can be equal to the PVI or other properties that refer to the injected water. In the instance of function variations, typical values of α also tend to change.

When finding the coefficient α Sw can be calculated for any PVI. For high PVI values, it is possible to arrive at the SOR value as shown in equation 2:

Sor=1−Sw(PVI) (Eq. 2)

In this instance, 350 was chosen as the hypothetical PVI terminal value, which, when applied to the Sw(PVI) function, returns the water saturation (Sw) at the residual oil saturation (SOR).

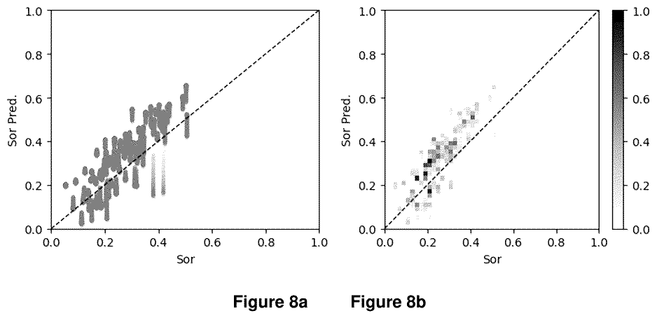

FIG. 7 shows the flow applied to determine SOR by the Sw(PVI) function, and FIGS. 8a-8b show the results of determining SOR by applying the Sw(PVI) method on the experimental Sw and PVI data and the comparison with the experimental SOR data.

In a second embodiment, the SOR value can be achieved by multiple Linear Regression between Sw (or Sxo at a well scale), SOR and PVI, which can be expressed by the equation:

Sor=b0+b1*Sw+b2*PVI (Eq. 3)
wherein:
   b0=intercept coefficient;
   b1, b2=estimated coefficients for the independent variables
   Sw=water saturation;
   Sor=residual oil saturation.

Reference values for the coefficients extracted from water injection test data on oil-saturated rock samples (core flood), are 0.695704, −0.819335 and 0.0011 for b0, b1 and b2, respectively. However, these values may vary for different oil fields.

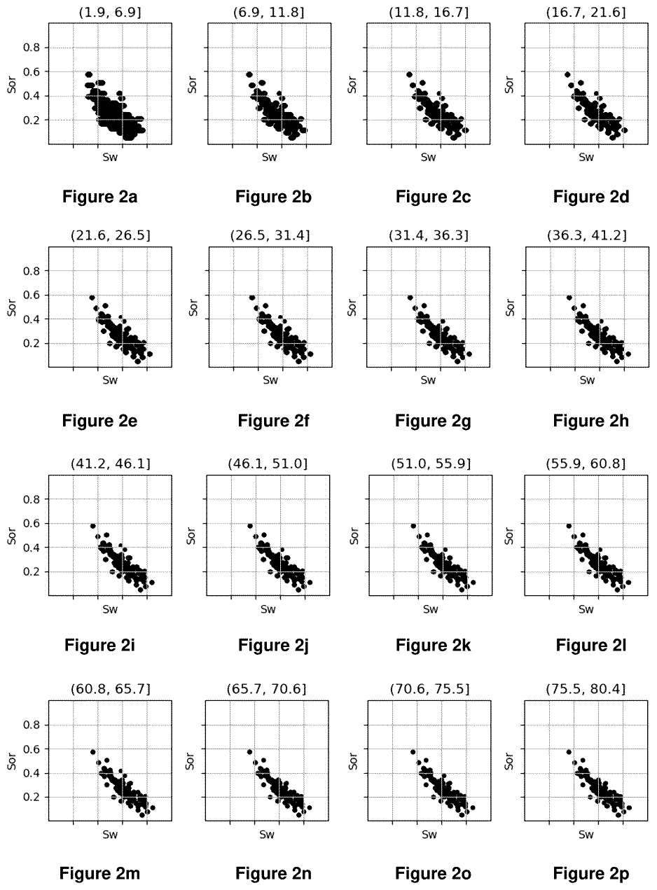

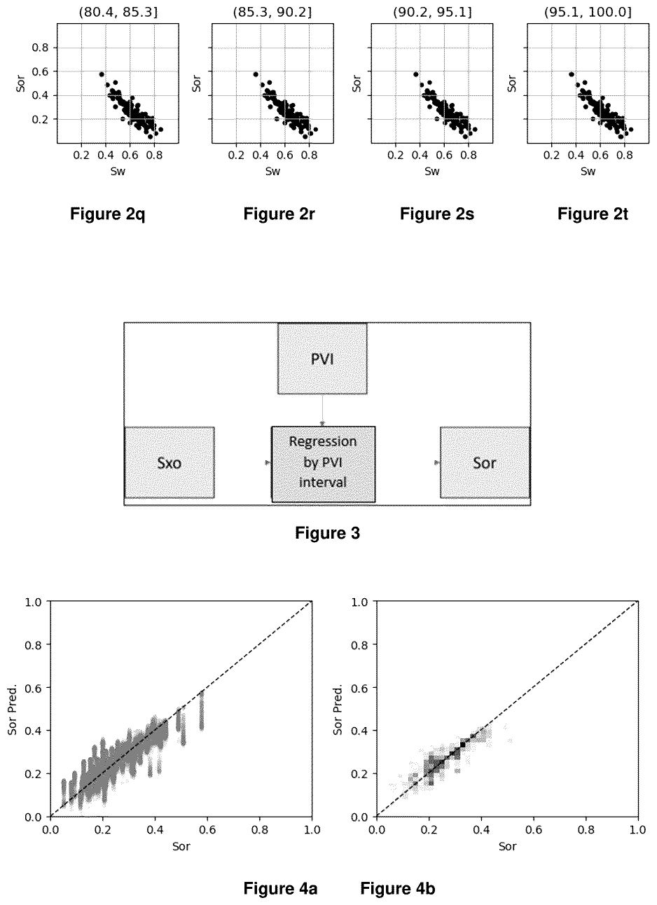

In a third embodiment, the SOR value can be obtained through linear regressions, non-linear regressions, machine learning and/or any other similar methods using the relationships between PVI, Sw with Sor. Such relationships are particularly useful, for example, in machine learning techniques, in which a known SOR value is used to find the SOR value when and where this value is unknown. When the Sw and SOR data are separated into PVI intervals, that is, for each water injection time point, it can be observed that the relationship between Sw and Sor is inversely linear and can be estimated by a simple linear regression. FIGS. 2a-2t exemplify this, and Table 1 shows the linear regression equation coefficients between Sw and Sor for each PVI interval. FIG. 3 shows the flowchart that summarizes the application of the method for the second and third embodiments.

Among the advantages of the described methods are: (1) the provision of a method for determining reservoir SOR at the well scale, (2) ease of application in petrophysical reservoir evaluation and reservoir engineering due to the practicality of the method; (3) determination of SOR with greater precision and accuracy, (4) economic improvement of reserve and oil production estimates in the reservoir, (5) reduction in the uncertainties of reserve and production estimates; and (6) contribution to the increase of reserves.

Examples of the Use of this Method in a Wellbore

FIGS. 2a-2t show scatter plots of Sw vs SOR for each PVI interval. Data obtained show the inverse correlation of the two variables. It is also noted that the narrower the PVI interval, the better the correlation between Sw and SOR, and the greater the PVI the better the correlation; so if PVI and Sw are known, SOR can be determined by a simple regression such as y=a·x+b.

Table 1 shows the linear equation parameters for each PVI interval. It is important to emphasize that the values in Table 1 are reference values for the analyzed samples and may differ for different reservoirs; the PVI intervals can even vary. Therefore, the essence of the method is the idea of estimating Sor by correlating Sw, Sor and PVI, not the coefficients and parameters of the regressions nor the PVI intervals themselves, and other regression methods can even be applied without affecting the protection granted by the present application.

TABLE 1
Parameters of the linear equation for each PVI interval
SOR = a · Sw + b

| | | |
|---|---|---|
| PVI interval | a | b |

| | | | |
|---|---|---|---|
| 1.9 | 6.9 | −0.81 | 0.68 |
| 6.9 | 11.8 | −0.84 | 0.73 |
| 11.8 | 16.7 | −0.85 | 0.75 |
| 16.7 | 21.6 | −0.86 | 0.76 |
| 21.6 | 26.5 | −0.86 | 0.76 |
| 26.5 | 31.4 | −0.86 | 0.77 |
| 31.4 | 36.3 | −0.87 | 0.78 |
| 36.3 | 41.2 | −0.87 | 0.78 |
| 41.2 | 46.1 | −0.87 | 0.79 |
| 46.1 | 51.0 | −0.87 | 0.79 |
| 51.0 | 55.9 | −0.88 | 0.79 |
| 55.9 | 60.8 | −0.88 | 0.80 |
| 60.8 | 65.7 | −0.88 | 0.80 |
| 65.7 | 70.6 | −0.88 | 0.80 |
| 70.6 | 75.5 | −0.88 | 0.80 |
| 75.5 | 80.4 | −0.89 | 0.81 |
| 80.4 | 85.3 | −0.89 | 0.81 |
| 85.3 | 90.2 | −0.89 | 0.81 |
| 90.2 | 95.1 | −0.88 | 0.81 |
| 95.1 | 100.0 | −0.90 | 0.82 |

FIG. 3 shows the flowchart of the proposed method and FIGS. 4a-4b show the result of SOR determination using the regression method in comparison with the experimental SOR measured in the relative permeability test in the laboratory.

Linear regression with an inverse correlation between Sxo and SOR discriminated for each range of injected water volume (PVI), allows determining the SOR.

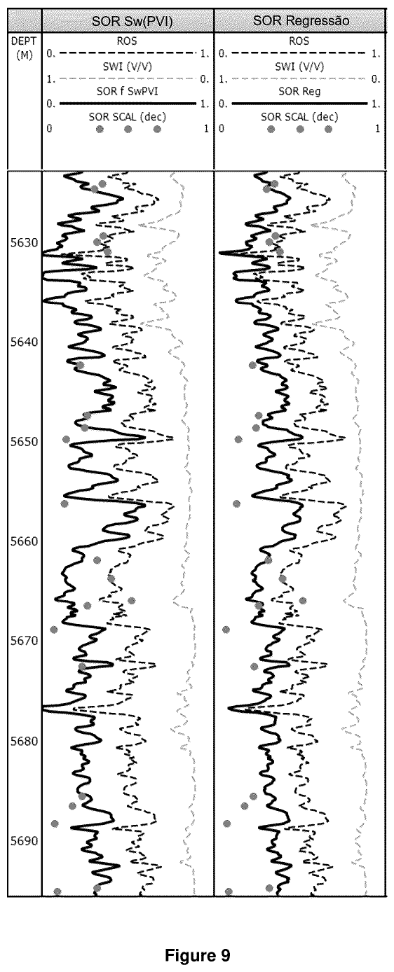

The methods according to the present invention have been successfully applied in real well bores, as seen, for example, in FIG. 9. It is worth highlighting that the data shown therein is not, in any way, limiting its scope, considering that possible additional alternatives exist. The two methods described in the present document, i.e., regressions and the Sw(PVI) function, were applied, thus estimating the SOR curves at well/log scale, which were compared with experimental SOR data (SCAL) and with the remaining oil saturation (ROS) estimated by Archie and the microresistivity profile.

The description of the present invention given so far should be considered only as a possible embodiment, and any particular features should be understood as being described to facilitate understanding. Therefore, they cannot be considered to limit the invention, which is limited only by the scope of the appended claims.

1. A method for determining residual oil saturation (SOR), wherein the method is expressed through the function Sw(PVI), from the following equations:
a) Obtaining Sw(PVI)
$$Sw ( PVI ) = \frac{{PVI}_{\sqrt{{PVI}^{a}}}^{a / 2}}{20 * a} + Swi \qquad ( Eq . 1 )$$
wherein:
PVI=injected pore volume;
Sw=water saturation;
Swi=irreducible water saturation, and
a=fitting coefficient of the function Sw(PVI).

2. The method for determining residual oil saturation (SOR), according to claim 1, wherein the parameter a ranges from 0.14 to 0.30.

3. The method for determining residual oil saturation (SOR), according to claim 1, wherein the SOR value is obtained using Equation 2:
Sor=1−Sw(PVI) for PVI>350 (Eq. 2).

4. The method for determining residual oil saturation (SOR), according to claim 1, wherein the SOR value is obtained by Multiple Linear Regression between Sw (or Sxo at the well scale), SOR and PVI, using equation 3:
Sor=b0+b1*Sw+b2*PVI (Eq. 3)
wherein:
b0=intercept coefficient;
b1, b2=estimated coefficients for the independent variables;
Sw=water saturation;
Sor=residual oil saturation.

5. The method for determining residual oil saturation (SOR), according to claim 1, wherein SOR is obtained using linear regressions, non-linear regressions or machine learning using the PVI, Sw properties and/or any other similar methods.

6. The method for determining residual oil saturation (SOR), according to claim 1, wherein the SOR value is obtained by any other similar mathematical method that presents variations of the Sw(PVI) function, wherein Sw is the dependent variable, and wherein the independent variable can be equal to the PVI or other properties that refer to the injected water.
